(19) United States
(12) Patent Application Publication
(10) Pub. No.: US 2012/0083621 A1
(43) Pub. Date: Apr. 5, 2012

(54) METHOD AND APPARATUS FOR PREPARING MULTIPLE ESTERS

(75) Inventors: HSIAO-PING HUANG, TAIPEI (TW); I-LUNG CHIEN, TAIPEI (TW); MING-JER LEE, TAIPEI (TW); I-KUAN LAI, TAIPEI (TW)

(73) Assignee: NATIONAL TAIWAN UNIVERSITY, TAIPEI (TW)

(21) Appl. No.: 12/938,251

(22) Filed: Nov. 2, 2010

(30) Foreign Application Priority Data
Sep. 30, 2010 (TW) 099133360
Sep. 30, 2010 (TW) 099133363

(51) Int. Cl. C07C 67/08; B01J 19/00

(52) U.S. Cl. 560/129; 422/187

(57) ABSTRACT

Disclosed is a method and an apparatus for manufacturing at least two esters. The method includes steps of: (a) reacting an acid and an alcohol mixture in a reactive distillation (RD) column to generate an alcohol-ester-water azeotrope, wherein the alcohol mixture includes at least two alcohols; (b) separating the alcohol-ester-water azeotrope as the organic mixture and water in a decanter; (c) purifying the organic mixture to generate the ester mixture in a stripper; and (d) performing the ester-ester separation on the ester mixture to generate at least two esters in a distillation column, wherein the number of at least two esters is the same with that of at least two alcohols. The unpurified ester mixture carrying heat can be refluxed into stripper for saving energy and enhancing the efficiency of purification.

### FIELD OF THE INVENTION

The present invention relates to a method for preparing esters and its apparatus. In particular, the present invention relates to a method for preparing multiple esters in a system (an apparatus).

### BACKGROUND OF THE INVENTION

Acetate esters are widely applied in the industries such as varnishes, ink, synthetic resins, adhesive agents and perfume. Ethyl acetate (EtAc), n-butyl acetate (n-BuAc) and isopropyl acetate (IPAc) are the common C₁ to C₄ alkyl acetates.

The traditional EtAc preparation method is made by feeding acetic acid (HAc) and ethanol (EtOH) into the reactive distillation (RD) column and performing esterification in the catalyst reaction, and the alcohol-ester-water azeotrope is obtained in the column top. However, the purity of the obtained ester is not high enough. Furthermore, the alcohol-ester-water azeotrope is cooled to be separated as water phase and organic phase in the decanter, and then organic phase is refluxed into the RD column for refinery distillation so that EtAc with higher purity is obtained.

In addition, there has another EtAc preparation method to link an RD column with a stripper. Although the EtAc product with higher purity can be harvested at the bottom of the stripper, it is necessary to fed the acid-alcohol-ester azeotrope at the column top into the RD column again and to retreat the alcohol-water mixture at the bottom of the RD column. Therefore, it needs to spend time, procedures and energy on the other intermediates, and the purity of EtAc is limited.

At meanwhile, in the conventional technology, only an acid and an alcohol are fed into a system to reproduce ester and water. When another ester product is desired, the reaction apparatus needs to be designed again and parameters need to be adjusted. It makes complexity and inefficiency on the construction of the reaction apparatus.

It is therefore attempted by the applicant to resolve the above situation encountered in the prior article.

### SUMMARY OF THE INVENTION

For overcoming the drawbacks in the prior art, in the present invention, a system (or an apparatus) is designed to esterify an acid with two or more alcohols to individually obtain two or more esters with high purity via “heat at reflux” of the intermediates (i.e. the ester mixture) for saving energy. Furthermore, water reproduced in the esterification also can be efficiently excluded from the ester mixture and the individual high-purify esters.

The first conception of the present invention provides a method for preparing at least two esters, including steps of: (a) reacting an acid with an alcohol mixture containing at least two alcohols to generate a first gas mixture; (b) cooling the first gas mixture to generate a first liquid mixture; (c) separating the first liquid mixture into a second liquid mixture and water; (d) purifying a first section of the second liquid mixture to generate an ester mixture; and (e) distilling the ester mixture to generate the at least two esters. A total number of the at least two esters equals to that of the at least two alcohols.

Preferably, the step (a) is performed in an RD column, and the method further includes a step (a0) of feeding the acid and the alcohol mixture into the RD column via a first reboiler. The acid in the RD column is refluxed into the first reboiler to be heated and then is fed into the RD column. The step (c) further includes a step (c1) of feeding a second section of the second liquid mixture into the RD column.

Preferably, the step (c) is performed in a decanter, the step (d) is performed in a stripper, and the first section of the second liquid mixture further generates a second gas mixture to be further cooled and fed into the decanter. In accordance with one embodiment, the step (e) is performed in a distillation column, and the step (e) further includes a step (e1) of refluxing the ester mixture into the distillation column via a second reboiler.

Preferably, in accordance with another embodiment, each of the at least two esters has a boiling point, and the step (e) further includes a step (e1) of refluxing the ester of a higher boiling point into the distillation column via a third reboiler. The method further includes a step (f) of refluxing a first fraction of the ester mixture in the distillation column to be further purified with the remaining first section of the second liquid mixture.

The second conception of the present invention provides an apparatus corresponding to the afore-mentioned preparation method, and the apparatus includes: an RD column reacting an acid with an alcohol mixture including at least two alcohols to generate a first gas mixture; a decanter separating a first liquid mixture condensed from the first gas mixture into a second liquid mixture and water; a stripper purifying a first section of the second liquid mixture to generate an ester mixture; and a distillation column distilling the ester mixture to generate the at least two esters. A total number of the at least two esters is equal to that of the at least two alcohols.

Preferably, the RD column further contains an ionic exchange resin for catalyzing the acid and the alcohol mixture. The acid includes acetic acid, and Each of the at least two alcohols has a carbon number ranged between 2 and 5.

Preferably, the apparatus further includes a first reboiler for feeding the acid and the alcohol mixture into the RD column, and the acid refluxes into the first reboiler to be heated and then is fed into the RD column. The apparatus further includes a second reboiler for heating the ester mixture, and a first part and a second part of the ester mixture respectively is fed into the distillation column and the stripper.

Preferably, the at least two esters includes a first ester with a first boiling point and a second ester with a second boiling point, the first boiling point is lower than the second boiling point, and the apparatus further includes a third reboiler heating the second ester.

Preferably, a first fraction of the ester mixture in the distillation column is refluxed into the stripper.

Preferably, the apparatus further includes a condenser for condensing the first ester, and a first portion of the first ester is fed into the distillation column.

The third conception of the present invention provides a method for preparing multiple esters, including steps of: (a) reacting an acid with an alcohol mixture containing multiple alcohols to generate a first mixture; (b) separating the first mixture to generate a second mixture; (c) purifying the second mixture to generate an ester mixture; and (d) distilling the ester mixture to generate the multiple esters having a total number equal to that of the multiple alcohols.

Preferably, a first section of the second mixture is purified into the ester mixture and a second section of the second mixture is further reacted in the step (a). A first part of the ester mixture is heated to be further purified in the step (c). The multiple esters at least have a first ester with a relatively lower boiling point and a second ester with a relatively higher boiling point, and a first portion of the first ester is heated to be further distilled in the step (d).

Preferably, the second mixture further generates a gas mixture, and the step (c) further includes steps of: (c1) cooling the gas mixture to generate a liquid; and (c2) feeding the liquid to the first mixture. The method further includes a step (e) of feeding back a first fraction of the ester mixture to be further purified with the remaining ester mixture.

The above objectives and advantages of the present invention will become more readily apparent to those ordinarily skilled in the art after reviewing the following detailed descriptions and accompanying drawings.

### BRIEF DESCRIPTION OF THE DRAWINGS

FIG. 1 depicts the flow chart showing the preparation of multiple esters according to the first preferred embodiment of the present invention.

FIG. 2 depicts the flow chart showing the preparation of multiple esters according to the first preferred embodiment of the present invention.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### The First Preferred Embodiment

The manufacturing apparatus of the multiple esters in the first preferred embodiment mainly includes an RD column, a decanter, a stripper and a distillation column. The acid (such as acetic acid (HAc)) is reacted with the alcohol mixture (such as ethanol (EtOH) and isopropanol (IPOH)) to generate the unpurified alcohol-ester-water azeotrope in the catalyst reaction in the RD column. The unpurified alcohol-ester-water azeotrope is fed to the decanter and separated to generate the organic phase and the water phase. The organic phase is fed into the stripper and purified as the ester mixture, and the remaining alcohol and water are recycled. The purified ester mixture then is fed into the distillation column to perform the ester-ester purification, so that the multiple esters (such as ethyl acetate (EtAc) and isopropyl acetate (IPAc)) are individually purified. According to the above method, the total number of the alcohols in the alcohol mixture is equal to that of the ester products. The reaction formula I of the aforementioned embodiment is listed as follows.

EtOH+IPOH+HAc→EtAc+IPAc+H₂O (Formula I)

The detailed preparation method of the multiple esters and the apparatus are described as follows.

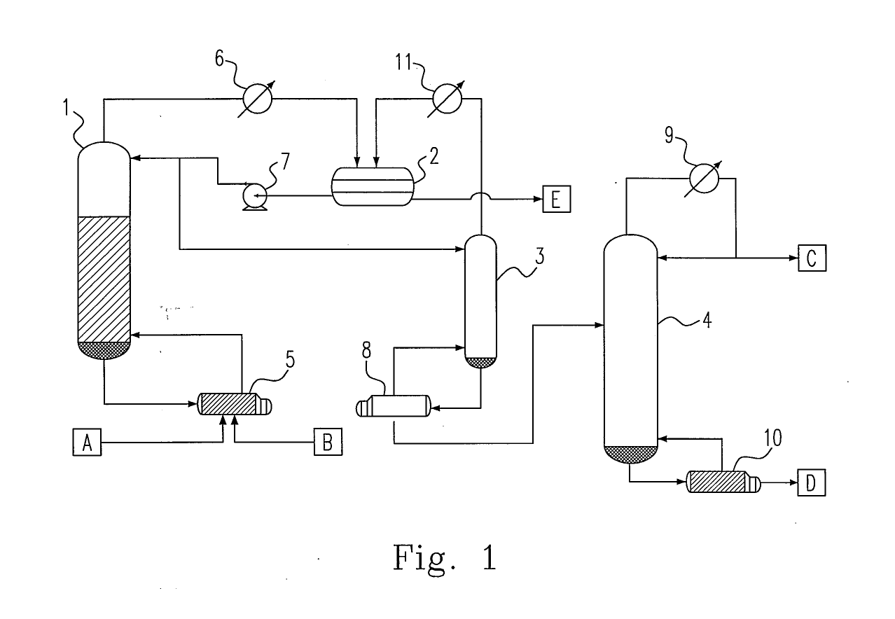

Please refer to FIG. 1, which depicts the flow chart showing the preparation of multiple esters according to the first preferred embodiment of the present invention. In FIG. 1, the acid A and the alcohol mixture B are fed into the RD column 1 via the reboiler 5. In the present invention, the acid A is HAc, and the alcohol mixture B contains the mixture of EtOH and IPOH. However, the acid A includes but not limit in HAc, and other acids also are applicable. The alcohol mixture B includes but not limit in EtOH and IPOH. Low carbon-numbered alcohol (C₂ to C₅) can be the reactants in the present invention. The ratio of each alcohol in the alcohol mixture B will decide the ratio of each prepared ester.

The acid A is reacted with the alcohol mixture B using the ionic exchange resin, Amberlyst 15 (Rohm and Hass), as the solid catalyst in the reaction portion (i.e. the middle portion) of the RD column 1, and the first gas mixture is obtained in the distillation portion (i.e. the top portion) of the RD column 1. Because of the boiling points of the reactants (HAc, EtOH and IPOH) and the products (EtAc, IPAc and H₂O) and the azeotropic point of the existing azeotrope, the first gas mixture obtained in the column top is the alcohol-ester-water azeotrope while the bottom portion of the RD portion 1 is the acid A with high purity. The first gas mixture is cooled as a first liquid mixture via a condenser 6. In the decanter 2, the first liquid mixture is separated as the water phase and the organic phase. The water phase is drained out in the form of the liquid water (product E). The first section of organic phase (i.e. the second liquid mixture) is fed into the stripper 3 via the pump 7 to further purify as a high-purity ester mixture while the second section of organic phase is refluxed to the top portion of the RD column 1. The unpurified alcohol-ester-water gas mixture is exported from the top of the stripper 3 and fed into the decanter 2 via the condensation in the condenser 11.

The high-purity ester mixture is exported from the stripper 3, heated in the reboiler 8, and then a first part of the ester mixture is fed into and distilled in the distillation column 4. A second part of the ester mixture with high purity is fed into the stripper 3 due to the heat energy to enhance the efficiency of purification. The first ester (EtAc with boiling point of 77.20° C.) with the relatively lower boiling point is obtained in the top portion of the distillation column 4. The first ester is cooled as the condensed first eater C via the condenser 9, and a part of the first ester also can be refluxed into the distillation column 4 to enhance its purity. The second ester (IPAc with boiling point of 88.52° C.) with the relatively higher boiling point is obtained in the bottom portion of the distillation column 4, and the second ester is heated via the reboiler 10. A first portion of the second ester is refluxed to the distillation column 4 to enhance its purity, and the remaining second ester is harvested as the second ester D.

In the example performed based on the first preferred embodiment, the molar ratio of the acid A (HAc) to the alcohol mixture B (including EtOH and IPOH) is 1:1.05. As to HAc, its pressure is 2 atm, and flow rate is 99.4 kmole/hr. As to the alcohol mixture B, its pressure is 2 atm, flow rate is 100 kmole/hr, and the molar ratio of EtOH to IPOH is 1:1. The reactants are reacted in the RD column 1 (diameter: 5.0073 m) via the reboiler 5 (reboiler duty: 18,789 kw), and the first gas mixture (Table 1) is exported from the column top. After the first liquid mixture is separated, a second section of the organic phase is refluxed to the RD column 1 at the reflux ratio of 5.7836, and the first section thereof is fed into and purified in the stripper 3 (diameter: 1.4609 m). The unpurified alcohol-ester-water gas mixture is condensed in the condenser 11, and the condensed mixture (Table 1) is fed into the decanter 2. Water E (flow rate: 19 kmole/hr, molecular ratio: 0.9938) is separated from the decanter 2.

The purified ester mixture is fed into the reboiler 8 (reboiler duty: 2,230 kw), and the heated ester mixture (Table 1) then is fed into and distillated in the distillation column 4 (diameter: 3.757 m). The obtained first ester (EtAc, Table 1) passes the condenser 9, and a part of the first ester is refluxed into and distillated in the distillation column 4 at the reflux ratio of 27.5. The obtained second ester (IPAc, Table 1) is heated via the reboiler 10 (reboiler duty: 12,262 kw), and a first portion of the second ester is refluxed into and distillated in the distillation column 4.

TABLE 1
The results of the intermediates in each stage and the end
products in the preparation method of the multiple esters

| | Flow rate | Molar ratio | | |
|---|---|---|---|---|
| Name | (kmole/hr) | IPAc | EtAc | H₂O |
| First gas mixture | 1201.4 | 0.3501 | 0.4372 | 0.2005 |
| First section of the | 1002 | 0.3703 | 0.4744 | 0.1412 |
| organic phase | | | | |
| Heated ester mixture | 99.3465 | 0.4976 | 0.4975 | 3.8011 × 10⁻⁵ |
| First ester (EtAc) | 49.4179 | 0.0002 | 0.99 | |
| Second ester (IPAc) | 49.9286 | 0.99 | 0.01 | |
| Condensed mixture | 73.9008 | 0.2158 | 0.0050 | 0.3310 |

It can be known from Table 1 that the ester mixture exported from the stripper 3 contains the equal amounts of EtAc and IPAc and contains a few water, and high-purity EtAc and IPAc are finally obtained. Therefore, in the present invention, the esterification of two mixed alcohols with acid are effectively performed to purify as two high-purity esters.

In addition, in the first preferred embodiment of the present invention, the reboiler 8 is configured to process the heat integration. The second part of the heated ester mixture is fed to the stripper 3 while the first part thereof is fed into the distillation column to proceed the ester-ester purification. Thus, about 10% energy is saved.

### The Second Preferred Embodiment

The manufacturing apparatus, the procedures and the reaction formula of the multiple esters in the second preferred embodiment are similar to those in the first preferred embodiment. The difference between the first and the second preferred embodiments lies in that, in the second preferred embodiment, the vapor ester mixture which is not separated as the individual esters but carries heat energy in the distillation column is fed into the stripper to supply energy on the purification of the unpurified ester mixture.

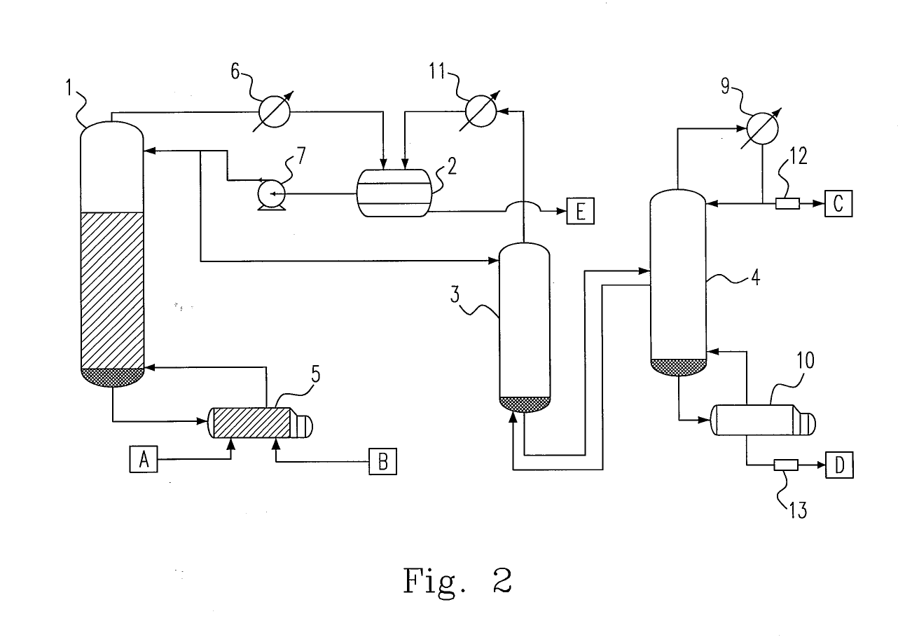

Please refer to FIG. 2, which depicts the flow chart showing the preparation of multiple esters according to the second preferred embodiment of the present invention. In FIG. 2, the steps of esterification, condensation, separation and purification are the same with the steps in the first preferred embodiment, and the detailed illustration of these steps are omitted.

In the second preferred embodiment, the high-purity liquid ester mixture is transported from the stripper 3 to the distillation column 4 via the pipeline to perform distillation, and the unpurified high-purity vapor ester mixture with heat energy is transported from the distillation column 4 to the stripper 3 via another pipeline to perform purification, so that efficiency of purification is enhanced and energy is saved. Similarly, the first ester (EtAc) obtained in the top portion of the distillation column 4 is cooled as the condensed first eater C via the condenser 9 and the distilling still 12 sequentially, and a part of EtAc is refluxed into the distillation column 4 to enhance its purity. The second ester (IPAc) obtained in the bottom portion of the distillation column 4 is heated via the reboiler 10. A first portion of the second ester is refluxed to the distillation column 4 to enhance its purity and provides the energy for ester-ester purification, and the remaining second ester is harvested as the second ester D via the distilling still 13.

In the example performed based on the second preferred embodiment, the molar ratio of HAc to EtOH—IPOH mixture is 1:1.05, wherein HAc has pressure of 2 atm and flow rate of 99.37 kmole/hr, EtOH—IPOH mixture has pressure of 2 atm and flow rate of 100 kmole/hr, and the molar ratio of EtOH to IPOH is 1:1. The reactants are reacted in the RD column 1 (diameter: 4.3606 m) via the reboiler 5 (reboiler duty: 15,237 kw), and the first gas mixture (alcohol-ester-water azeotrope, Table 2) is exported from the RD column top. After the first liquid mixture is separated, a second section of the organic phase is refluxed to the RD column 1 at the reflux ratio of 5.8837, and the first section thereof is fed into and purified in the stripper 3 (diameter: 1.2147 m). The unpurified alcohol-ester-water gas mixture is condensed in the condenser 11, and the condensed mixture (Table 2) is fed into the decanter 2. Water E (Table 2) is separated from the decanter 2.

The purified liquid ester mixture (Table 2) is fed into and distilled in the distillation column 4 (diameter: 3.4977 m). The obtained first ester (EtAc, Table 2) passes the condenser 9, and a part of the first ester is refluxed into and distillated in the distillation column 4 at the reflux ratio of 20.01, and another part thereof is harvested via the distilling still 12. The obtained second ester (IPAc, Table 2) is heated via the reboiler 10 (reboiler duty: 10,890 kw), a first portion of the second ester is refluxed into and distillated in the distillation column 4, and a second portion thereof is harvested via the distilling still 13.

The unpurified ester mixture in the distillation column 4 carries kinetic energy because of its heat, and refluxes into the stripper 3 in the form of vapor. The unpurified vapor ester mixture provides energy with the unpurified ester mixture for purification. The energy for the system can be saved, and the unpurified ester mixture is purified as the ester mixture with higher purity to be benefit for the further ester-ester purification procedure.

It can be known from Table 2 that the ester mixture exported from the stripper 3 contains the equal amounts of EtAc and IPAc and contains a few water, and high-purity EtAc and IPAc are finally obtained. Therefore, in the present invention, the esterification of two mixed alcohols with acid are effectively performed to purify as two high-purity esters.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 2
The results of the intermediates in each stage and the end products in the preparation
method of the multiple esters

| | Flow rate | Molar ratio | | | | | |
|---|---|---|---|---|---|---|---|
| Name | (kmole/hr) | IPAc | EtAc | HAc | EtOH | IPOH | H₂O |
| First gas mixture | 1127.07 | 0.3692 | 0.4343 | 0.0007 | 0.0030 | 0.0045 | 0.1883 |
| [symbol] st section of the other organic | 927.7 | 0.3953 | 0.4739 | 0.0007 | 0.0035 | 0.0049 | 0.1218 |
| phase | | | | | | | |
| Purified ester mixture (liquid) | 279.7267 | 0.5481 | 0.4474 | 0.0004 | 0.0008 | 0.0023 | 0.0010 |
| Ester mixture (vapor) | 180 | 0.5777 | 0.4207 | 1.4999 × 10⁻⁵ | 0.0003 | 0.0011 | 0.0003 |
| First ester (EtAc) | 49.9185 | 0.0002 | 0.9823 | 2.95 × 10⁻²⁹ | 0.0034 | 0.0093 | 0.0049 |
| Second ester (IPAc) | 49.8082 | 0.9901 | 0.0079 | 0.0020 | 2.8328 × 10⁻¹² | 2.5015 × 10⁻¹⁵ | 1.3248 × 10⁻³⁵ |
| H₂O (E) | 99.6431 | 0.0008 | 0.0040 | 0.0004 | 1.7064 × 10⁻¹¹ | 0.0014 | 0.9934 |
| Unpurified alcohol-ester-water | 57.9449 | 0.2244 | 0.4366 | 2.708 × 10⁻⁵ | 0.0065 | 0.0052 | 0.3272 |
| gas mixture | | | | | | | |

[symbol]

indicates data missing or illegible when filed

What is claimed is:

1. A method for preparing at least two esters, comprising steps of:
(a) reacting an acid with an alcohol mixture comprising at least two alcohols to generate a first gas mixture;
(b) cooling the first gas mixture to generate a first liquid mixture;
(c) separating the first liquid mixture into a second liquid mixture and water;
(d) purifying a first section of the second liquid mixture to generate an ester mixture; and
(e) distilling the ester mixture to generate the at least two esters, wherein a total number of the at least two esters is equal to a total number of the at least two alcohols.

2. The method according to claim 1, wherein the step (a) is performed in a reactive distillation column, and the method further comprises a step (a0) of feeding the acid and the alcohol mixture into the reactive distillation column via a first reboiler.

3. The method according to claim 2, wherein the acid in the reactive distillation column is refluxed into the first reboiler to be heated and then is fed into the reactive distillation column.

4. The method according to claim 2, wherein the second liquid mixture further has a second section, and the step (c) further comprises a step (c1) of feeding the second section into the reactive distillation column.

5. The method according to claim 1, wherein the step (c) is performed in a decanter, the step (d) is performed in a stripper, and the first section of the second liquid mixture further generates a second gas mixture to be further cooled and fed into the decanter.

6. The method according to claim 1, wherein the step (e) is performed in a distillation column, and the step (e) further comprises a step (e1) of refluxing the ester mixture into the distillation column via a second reboiler.

7. The method according to claim 1, wherein the step (e) is performed in a distillation column, each of the at least two esters has a boiling point, and the step (e) further comprises a step (e1) of refluxing the ester of a higher boiling point into the distillation column via a third reboiler.

8. The method according to claim 7, wherein the ester mixture in the distillation column has a first fraction, and the method further comprises a step (f) of refluxing the first fraction to be further purified with the remaining first section of the second liquid mixture.

9. An apparatus for preparing at least two esters, comprising:
a reactive distillation column reacting an acid with an alcohol mixture comprising at least two alcohols to generate a first gas mixture;
a decanter separating a first liquid mixture condensed from the first gas mixture into a second liquid mixture and water;
a stripper purifying a first section of the second liquid mixture to generate an ester mixture; and
a distillation column distilling the ester mixture to generate the at least two esters, wherein a total number of the at least two esters is equal to a total number of the at least two alcohols.

10. The apparatus according to claim 9, wherein the reactive distillation column further comprises an ionic exchange resin for catalyzing the acid and the alcohol mixture, the acid comprises an acetic acid, and each of the at least two alcohols has a carbon number ranged between 2 and 5.

11. The apparatus according to claim 9 further comprising a first reboiler, wherein the acid and the alcohol mixture are fed into the reactive distillation column via the first reboiler, the acid refluxes into the first reboiler to be heated and then is fed into the reactive distillation column.

12. The apparatus according to claim 11 further comprising a second reboiler heating the ester mixture, wherein the ester mixture has a first part and a second part, the first part is fed into the distillation column, and the second part is fed into the stripper.

13. The apparatus according to claim 12, wherein the at least two esters comprise a first ester with a first boiling point and a second ester with a second boiling point, the first boiling point is lower than the second boiling point, and the apparatus further comprises a third reboiler heating the second ester.

14. The apparatus according to claim 13, wherein the ester mixture in the distillation column has a first fraction to be refluxed into the stripper.

15. The apparatus according to claim 13 further comprising a condenser for condensing the first ester which has a first portion being fed into the distillation column.

16. A method for preparing multiple esters, comprising steps of:
(a) reacting an acid with an alcohol mixture comprising multiple alcohols to generate a first mixture;
(b) separating the first mixture to generate a second mixture;
(c) purifying the second mixture to generate an ester mixture; and
(d) distilling the ester mixture to generate the multiple esters having a total number equal to that of the multiple alcohols.

17. The method according to claim 16, wherein the second mixture has a first section to be purified into the ester mixture and a second section to be further reacted in the step (a), and the ester mixture has a first part heated to be further purified in the step (c).

18. The method according to claim 16, wherein the multiple esters at least have a first ester with a relatively lower boiling point and a second ester with a relatively higher boiling point, and the first ester has a first portion being heated to be further distilled in the step (d).

19. The method according to claim 16, wherein the second mixture further generates a gas mixture, and the step (c) further comprises steps of:
(c1) cooling the gas mixture to generate a liquid; and
(c2) feeding the liquid to the first mixture.

20. The method according to claim 16, wherein the ester mixture has a first fraction, and the method further comprises a step (e) of feeding back the first fraction to be further purified with the remaining ester mixture.
